(19) United States
(12) Patent Application Publication
(10) Pub. No.: US 2019/0236120 A1
(43) Pub. Date: Aug. 1, 2019

(54) ELECTRONIC TEXT TO BRAILLE DEVICE

(71) Applicants: Eric Thomas Reid, Ann Arbor, MI (US); Katherine Isabella Porfirio, Worcester, MA (US); Nelson Tansu, Bethlehem, PA (US)

(72) Inventors: Eric Thomas Reid, Ann Arbor, MI (US); Katherine Isabella Porfirio, Worcester, MA (US); Nelson Tansu, Bethlehem, PA (US)

(21) Appl. No.: 15/884,311

(22) Filed: Jan. 30, 2018

(51) Int. Cl. G06F 17/22; A61F 9/08; G09B 21/00; G06F 15/02

(52) U.S. Cl. CPC G06F 17/2223; G06F 15/025; G09B 21/004; A61F 9/08

(57) ABSTRACT

At present, a vast number of technologies are designed specifically for users that are able to employ the use of sight. Additionally, many products designed to allow the visually impaired to use these technologies are expensive, and often not easy to use. In order to expand the benefits of technology to the visually impaired, we have invented an Electronic Braille Interface that can connect to any device with a USB port (laptops, cell phones, e-readers, etc.) and effectively translate ASCII characters from the device into physical Braille characters. The Braille text will be displayed on the Electronic Braille Interface though moveable pins that, in groups of six, are able to display different Braille characters in the same fashion as normal, stationary Braille text. The user will be able to feel the pins and interpret the Braille characters. The pins are raised through a magnetic write head, which moves from letter to letter through the use of a motorized positioning system in order to raise the correct pins for each character. The pins are held in their up or down states through a system of springs and magnets. This allows a very small set of motors to easily write all of the characters on the display. A pin is held in the up position through the attraction of a magnet on the bottom of the pin with an iron layer embedded into the screen. The spring is in equilibrium when the pin is in the down position, and is compressed when the pin is in the up position; so that it can effectively hold the pin in the down position by default. When the write head reaches the pin, it applies a current to a coil directly underneath the pin, which will produce enough magnetic force to make the pin switch to the up position. A bar magnet resets all of the pins simultaneously by moving across the entire device and creating a magnetic force that causes all of the pins to reset to the down position. Additionally, a software program will be installed on any device connecting to the Electronic Braille Interface in order to write, erase, and control the pins. Our Electronic Braille Interface will be low-cost, lightweight, and able to integrate well into existing devices. Future generations will include the capability of drawing images, creating interactive displays, and allowing the user to play games (similar to the games that people often play on their cell phones or tablets).

### FIELD OF THE INVENTION

The present invention relates generally to the field of electronic displays for the visually impaired, and more specifically to portable and refreshing displays.

### BACKGROUND OF THE INVENTION

In the United States, approximately 2.3% of the population, or 7.3 million people, report a visual disability National Federation of the Blind, “Blindness Statistics”. 2016. nfb.org/blindness-statistics. These people are largely excluded from the target market of many different indispensable technologies that rely on the user's sight, such as laptops, cell phones, and e-readers. Furthermore, the current solutions that enable the visually impaired to use such technologies are often expensive and cumbersome. Given the statistically low income of visually impaired individuals, and the large percentage of visually impaired individuals living below the poverty line National Federation of the Blind, “Blindness Statistics”. 2016. nfb.org/blindness-statistics; it appears that expensive solutions that enable the visually impaired to use these indispensable technologies are simply not feasible in many cases. To remedy this problem, we have invented a portable Electronic Braille Interface capable of translating any text on a USB-enabled device into physical Braille characters. While various Braille displays already exist in various forms, our device is cheaper, lighter, and requires significantly less power than the competitive devices. The market for our device will not only consist of individuals with visual impairments, but also of institutions that are committed to complying with disability laws (such as libraries, schools, government institutions, and other public places), as disability laws mandate that these institutions make reasonable accommodations for those with disabilities.

### SUMMARY OF THE INVENTION

In accordance with the present invention, the Electronic Braille Interface uses a write head component. Instead of controlling each of the pins for every character separately, the write head component writes one character at a time. Current coils apply a magnetic force to a magnet on the bottom of each pin that forces the pin from the down to the up position. The pin is held up by the attraction between the magnet and an iron layer inside the display. The write head proceeds from character to character, line to line, until it has covered the entire reading area. When the user is done reading the displayed page, they can press a next page button. This causes a bar magnet to move up and down the device, pulling all of the pins downward and enabling the write head to return back to its starting point and write the next page. These processes can happen quickly because the write head only needs to move the pins 0.5 mm, the Braille standard text height.

### BRIEF DESCRIPTION OF THE DRAWINGS

In the drawings:

FIG. 1: Write head from top (left) and side views (right)

FIG. 2: Top-down view of write head and motor assembly, layer per motion direction

FIG. 3: Braille cell with dimensions

FIG. 4: Cross section of screen with springs and iron layer

FIG. 5: Top down view of the device

FIG. 6: Data flow between device and display

### DETAILED DESCRIPTION OF THE INVENTION

The objective in designing the write head is to create a device that can apply enough force to the pins to move them, while also using a very low amount of power. The necessary force is determined by the amount of force expected from a finger while reading the Braille, as the magnet must be attracted to the iron core by this much force. To calculate the amount of force expected by a finger, we investigated the guidelines for switches, as documented by Department of Defense, “Human Engineering Design Data Digest”. Human Factors Standardization Subtag. April, 2000. The Department of Defense concluded that between 2.8 N to 16.7 N is exerted for switch resistance. We assumed that the user would not be exerting the full 16.7 N in reading Braille, and decided to use a slightly more conservative 10 N force requirement for our calculations. To find the pressure from a finger, we measured the area of a small finger and concluded that a finger could exert pressure on the order of 100 kPa. Since the Braille standard pin diameter is 1.44 mm, the Braille pins on the Electronic Braille Interface must have a force of 0.16 N in their respective pin states. As this is quite a lot of magnetic force to be generated from a simple wire coil, we placed several magnets on the bottom of the write head to provide a baseline force. Consequently, the wire coil only needs to provide a small amount of additional force to set the pin to the up position. Each position on the write head has a magnet underneath it, so that all of the pins in one character are slightly raised when the write head is directly underneath them, due to the force between the two permanent magnets. The pins are not raised enough by this force alone to cause the pin magnets to be attracted to the iron layer embedded in the device. Consequently, if no current is applied, the pins are pulled back down by the springs after the write head moves on. If current is applied to the pins, however, enough force will be applied to the pins that they will move up far enough that their magnets will be attracted to the iron layer embedded in the device. FIG. 1 shows the write head from a top and side view, with the bottom of the pin shown in the side view.

Simulations of the interactions between the two magnets and the wire coil have indicated that there is a great deal of design flexibility. Due to the strong interaction between the magnets, they must be placed apart at a distance great enough to reduce the amount of applied force to below the force threshold of 0.16 N. A very conservative design puts 3 mm of space between the bottom of the pin and the top of the write head, with 5 turns of 0.5 oz copper wire being used to apply the electromagnetic forcing field. All 6 coils would draw around 60 μW of power, which is much lower than using motors or only the current coils to provide the force. The prototype device will likely have a larger write head-pin separation and use slightly more current to be easier to manufacture. However, the two-magnet method is flexible enough to allow a manufacturer to easily design the system to meet their specifications.

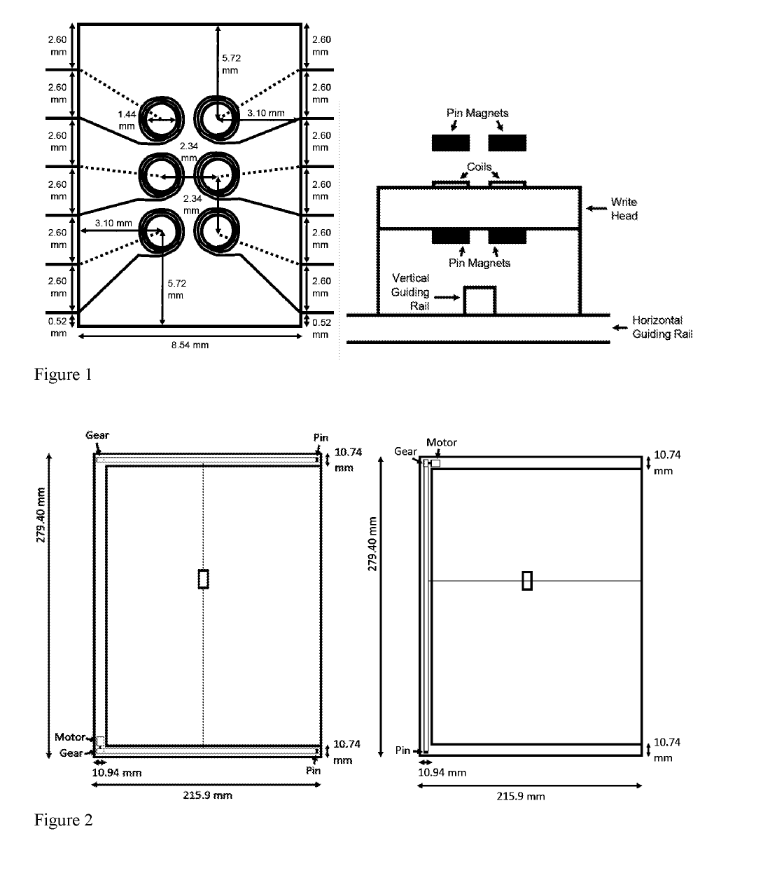

In order to move the write head from character to character, a two motor chain system is employed. The write head sits on top of two rectangular perpendicular holes. Beams are threaded through these holes, which are attached to the chains on the sides of the device. Rotating gears allow the chains to move back and forth, with their movement controlled by motors embedded in the corners of the screen. FIG. 2 shows the top-down view of this mechanism. By allowing the write head to slide on either beam freely, precise motion can be achieved through controlling how much each motor turns. Each motor turn moves the write head by a specific, known amount which is calculated by using the radius of the gear. A microcontroller controls each of the motors, which enables the write head to be moved from character to character as desired.

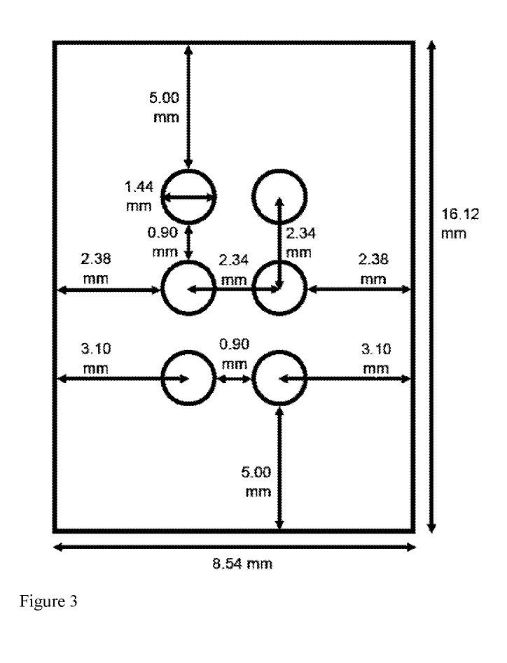

Braille has been standardized internationally in terms of character size and cell size Braille Authority of North America, “Braille Signage Guidelines”. 2014. We adopted these dimensions in order to allow the Electronic Braille Device to be easily integrated into the lives of the users. FIG. 3 shows the dimensions for one character, showing all 6 character pins from a vertical view. These dimensions determine the dimensions for the display and the internal mechanisms involved in making the device operate properly. By using these standard Braille dimensions in our design, we ensure that individuals who can already read Braille will not have to learn a new language or use different sizes in order to read the screen on the Electronic Braille Interface.

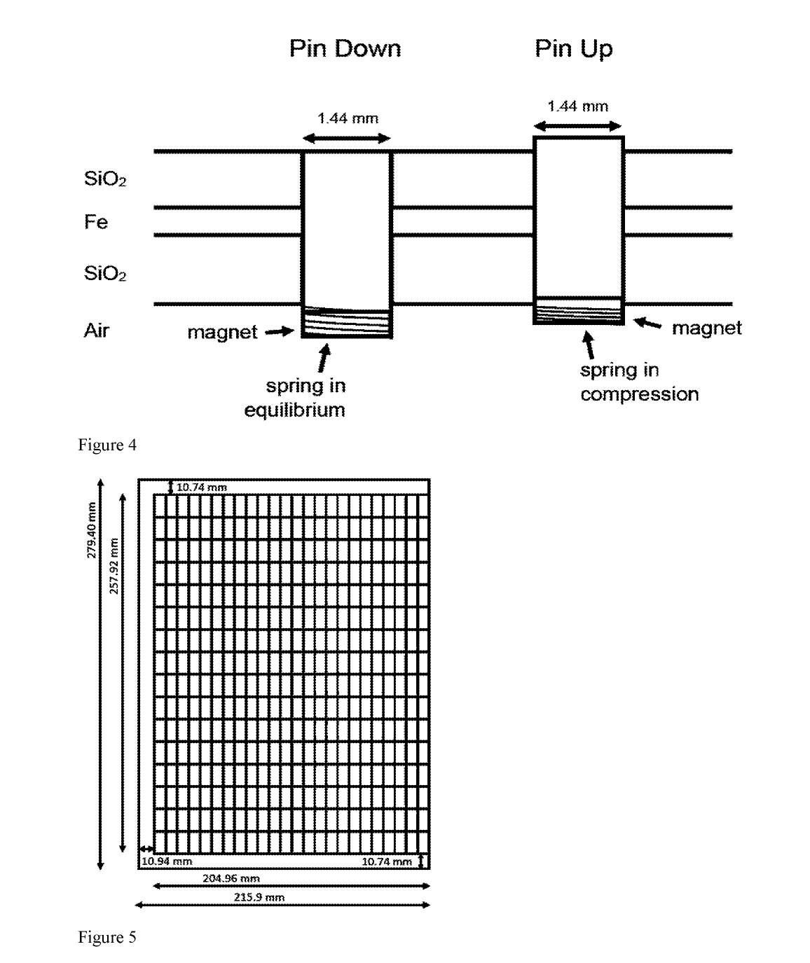

In order for the write head to be able to move around after setting the pins in the up or down position, the pins must be able to stay in a stable up or down position after being set by the write head. Therefore, two counteracting forces must neutralize each other at the up and down positions. The easiest force to use would be gravity, but due to the small pin size, the gravitational force is negligible. Furthermore, it would not be practical for the Braille pins to change states if the device is held upside-down. Therefore, a spring and an iron layer are used to hold the pin in the down and up positions, respectively. FIG. 4 shows the structure in cross section. The spring is attached to the bottom of the pin and applies force downward; the magnet used in the writing process is magnetically attracted to a thin iron layer in the screen to hold the pin up. A primary design constraint is balancing these two forces properly. This is especially difficult because the magnetic force is inversely proportional to the square of the vertical distance, while the spring force is proportional to the stretched length.

In a properly designed screen system, a pin will have two stable positions: one in the up position, and one in the down position. The maximum force applied must be greater than the expected force from a finger; otherwise, the user may inadvertently force the pins down while reading the Braille. Since this system requires no power to hold the pins up or down, however, the overall power draw is extremely low, as power is only expended during the writing process and during the resetting process. Most of the time the device is in use is spent by the user reading the Braille text, which requires no action from the write head. Consequently, the entire system will consume an extraordinarily low amount of power.

The screen itself is designed to be a full 8.5″×11″ page, which can fit 24 standard Braille characters across and 16 standard Braille characters down. In order to embed the motors for writing motion, and to give the user a place to hold the device, bezels slightly more than 10 mm will be placed on the top, bottom, and left sides of the device. It may be more aesthetically pleasing to put the bezels around the entire device, but this is a design decision that is not critical to device operation. FIG. 5 shows the top-down view of the screen, with each of the Braille characters shown as a box. As the intended user is visually impaired, the physical appearance of the device does not matter as much as it would for a normal e-reader. The materials used for the device, however, are important. The surface materials must be wear-resistant, and many plastics easily achieve this goal. The pins will likely be metal in order to provide a contrast between the pins and the rest of the device material.

In order for the Electronic Braille Device to be able to display multiple pages of text, it must be capable of resetting the pins before writing a new page. Theoretically, a large enough negative current in the write head could overpower the pins in every single character. A better solution, however, is to pass a large bar magnet back and forth over all of the pins, which would force them downward. This requires less power, as the only power consumption is with the driving motors. Since the reset bar must operate independently of the write head, a different set of motors and guiding rails must be put into the device. Unlike the write head, only one direction of motion is required, as the bar magnet will move across the width of the characters and pull all of the magnets down simultaneously. The bar will sweep back and forth, which allows two opportunities to pull the magnets down. It is unlikely that two passes will be required to pull all of the pins down, but the double-pass system will ensure that all of the pins are reset to the down position before the write head begins again. When the write head is writing or when the user is reading, the bar magnet is stored inside a side bezel, waiting to be called into action.

By using a forcing mechanism that is primarily passive, power consumption is lowered significantly. Additionally, due to the great strength of the magnets used on the pins (magnetic remanence over 1.2 T), the bar magnet can be relatively weak and still pull all of the pins down successfully. The double pass system ensures that if, by some small chance, a pin is not pulled down on the first sweep, it will be pulled down on the second sweep. This improves the long-term robustness of the system.

Each of the systems described above are integrated vertically, with the display situated on top, the write head situated below the bottoms of the pins, and the motorized driving system attached to the bottom of the write head. A microcontroller attached to the back of the system controls the motors, which control the write head, in order to make the device operate properly. The microcontroller also does communication interfacing through a USB connection with the device connected to the Electronic Braille Interface. This microcontroller is very small, as it does not have many computational requirements: Its primary role is to interface with the USB and control the driving motors.

The completed device will be somewhere between 2-3 cm thick, depending on the final design choices. This is thicker than most electronic devices, but the physical nature of Braille forces the thickness to be greater. Additionally, the user will likely be gripping the device for stability, instead of gently cradling in hand like a standard electronic device.

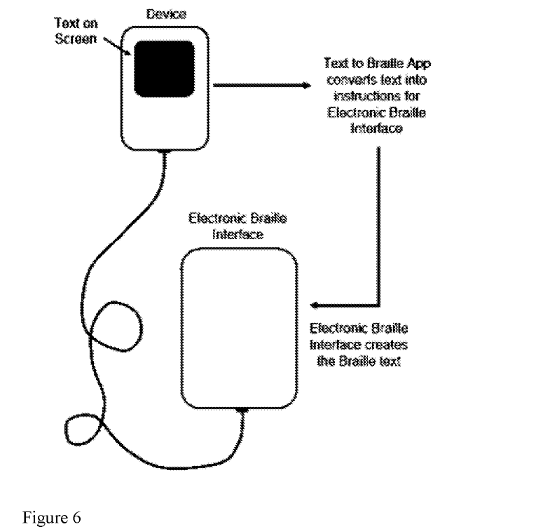

In addition to the physical Braille display, an application is downloaded on the primary electronic device, which converts the text on the screen into instructions that are sent to the Electronic Braille Interface. By doing the conversion from text to Braille on the primary device, the display itself can focus solely on displaying the text. Additionally, the application will be downloadable on a range of devices, and the output over the USB connection will be standardized. As a result, the display does not have to interface to all devices; it just needs to interface with the USB connection and the expected Braille communications. FIG. 6 shows the data flow in the whole system, detailing where the data is converted and transferred between the primary electronic device and the Braille display. This setup will allow software updates to enable pictures and other languages to be displayed on the Electronic Braille Interface.

In order to enable visually impaired individuals in our society to have greater access to the same technologies that we use every day, there is a need for a cheap, low-power electronic Braille display. Our invention accomplishes this by using many passive magnetic components to hold the Braille pins in their up and down positions, and to reset all of the pins on the screen. A single character write head applies additional magnetic fields and a small current to push the pins up as required to write characters. Overall, the system primarily only uses power to move the write head and the reset magnet. Additionally, very small amounts of power are used to write each individual pin and to power the microcontroller. The entire device is powered off of USB, with no batteries required.

The text is converted to Braille characters on the device that has the text, which enables the Electronic Braille Interface to have less computational power on board. This makes the Electronic Braille Interface both lighter and more power-efficient. Having software on the primary electronic device enables the system to be easily ported between different devices without having to change hardware. Additionally, software upgrades can enable additional languages, pictures and other features to be displayed without changing the physical device at all.

Future models of the Braille display could include the capability to form pictures by raising various pins in order to display an image that can be felt. The software on the primary device would send the picture over by character, in the same fashion that it sends over text. Further development could incorporate user feedback through touch screens or buttons, enabling electronic games to be played on the device. This would be similar to the ways in which modern electronics are used for a wide variety of games. Currently, these games are largely unavailable to the visually impaired, and future renditions of our technology would enable this form of recreation to reach these people.

By using many passive components and utilizing a single character write head, our display consumes significantly less power than existing competitors, and is cheaper due to the lack of individual drive components. Combined with the possibility of image display and live user feedback, our device offers a method to revolutionize the way the visually impaired are able to use electronics that many take for granted.

What is claimed is:

1. A device for assisting people with vision disability—referred as Braille device, said device comprising:
refreshable information providing functionality, a movable write head which moves across the display and writes each Braille character one at a time, a bi-stable mechanical holding system to hold pins either up or down after writing, a magnetic reset bar to pull all pins down, and the necessary motors and control electronics to drive the system.

2. The device of claim 1 wherein the write head uses inductive coils to move pins up.

3. The device of claim 1 wherein the bi-stable mechanical holding system uses Belleville washers.

4. The device of claim 1 wherein the bi-stable mechanical holding system is a balance of magnetic and spring forces.

5. The device of claim 1 wherein the software communication and control are handled by a handheld tablet device, smart phone, or laptop computer.

6. The device of claim 2 wherein the spring force is replaced with the gravitational force of the pin to the earth.

7. An interactive device for assisting people with vision disability—referred as Braille device, said device comprising:
Refreshable information providing functionality, interactive feedback response from users, a movable write head which moves across the display and writes each Braille character one at a time, a bi-stable mechanical holding system to hold pins either up or down after writing, a magnetic reset bar to pull all pins down, and the necessary motors and control electronics to drive the system.

8. The device of claim 7 wherein the write head uses inductive coils to move pins up.

9. The device of claim 7 wherein the bi-stable mechanical holding system uses Belleville washers.

10. The device of claim 7 wherein the bi-stable mechanical holding system is a balance of magnetic and spring forces.

11. The device of claim 7 wherein the software communication and control are handled by a handheld tablet device, smart phone, or laptop computer.

12. The device of claim 8 wherein the spring force is replaced with the gravitational force of the pin to the earth.

13. An interactive device with storage capability for assisting people with vision disability—referred as Braille device, said device comprising:
Refreshable information providing functionality, interactive feedback response from users, built-in storage capability, a movable write head which moves across the display and writes each Braille character one at a time, a bi-stable mechanical holding system to hold pins either up or down after writing, a magnetic reset bar to pull all pins down, and the necessary motors and control electronics to drive the system.

14. The device of claim 13 wherein the write head uses inductive coils to move pins up.

15. The device of claim 13 wherein the bi-stable mechanical holding system uses Belleville washers.

16. The device of claim 13 wherein the bi-stable mechanical holding system is a balance of magnetic and spring forces.

17. The device of claim 13 wherein the software communication and control are handled by a handheld tablet device, smart phone, or laptop computer.

18. The device of claim 14 wherein the spring force is replaced with the gravitational force of the pin to the earth.
